(19) United States
(12) Patent Application Publication
(10) Pub. No.: US 2024/0060108 A1
(43) Pub. Date: Feb. 22, 2024

(54) LOW LIPID CONTENT OAT PROTEIN COMPOSITION WITHOUT TRACES OF ORGANIC SOLVENT OR SURFACTANT

(71) Applicant: ROQUETTE FRERES, Lestrem (FR)

(72) Inventor: Kerry CAMPBELL, Geneva, IL (US)

(21) Appl. No.: 18/259,613

(22) PCT Filed: Jan. 4, 2022
(86) PCT No.: PCT/EP2022/025000

§ 371 Date: Jun. 28, 2023

(30) Foreign Application Priority Data
Jan. 4, 2021 (EP) 21305001.6

(51) Int. Cl. C12P 21/00

(52) U.S. Cl. CPC C12P 21/00

(57) ABSTRACT

The invention pertains to the field of oat protein compositions and production method thereof. In particular, the present invention is directed to an oat protein composition having low lipid content and which does not contain traces of organic solvent or surfactant and to the production method thereof.

### TECHNICAL FIELD

The invention pertains to the field of oat protein compositions and production method thereof. In particular, the present invention is directed to an oat protein composition having low lipid content and which does not contain traces of organic solvent or surfactant and to the production method thereof.

### BACKGROUND ART

Oats are a well-known source of a wide variety of useful products. Examples of such products are flour, starch, protein isolate and concentrate, protein-enriched flour, bran, gum and oil. Traditional techniques used in the cereal grain processing industry are frequently difficult to use with oats because of process problems relating to the presence of lipids in the oats. Moreover, unless the oats are de-oiled prior to milling, milling processes would result in the formation of flour and protein fractions containing lipids, which may result in the development of rancidity on storage of the flour and protein.

The most widely used technique consists in a first de-oiling step, carried out with the help of organic solvents like hexane or ethanol. The person skilled in the art is aware for example of EP0051943 from DUPONT which teaches the use of aliphatic hydrocarbon solvent to remove lipids from oat flours. Main drawbacks of such technologies are industrial use of organic solvent, associated explosion risks and spoilage, and residual levels of lipids in final products.

Such risks seem so important that the main current commercial product called PROATEIN® is currently produced without de-oiling. EP1706001 is only based on the use of amylases and centrifugal separation. As disclosed in the example part, such a process leads to a composition having a lipid content above 10% by weight based on total weight.

To address these drawbacks, some alternative processes have been recently proposed. Such processes are based on the use of supercritical CO₂. The person skilled in the art is aware of EP2120604 from VALTION TEKNILLINEN. However, to reach a high level of de-oiling, the flour needs to be processed and grinded, thereby leading to a mean particle size of protein below 10 microns (see paragraph 0047 of EP2120604). This process leads to a superfine size protein powder which is not desirable in some applications, but also which is difficult to handle in industrial plants, mainly due to dust formation and explosion hazard. Another major industrial problem linked to particles having a size below 10 microns is that the cyclone and filtration systems needed to recover such small particles are expensive and difficult to operate efficiently and/or effectively.

The document Brückner-Gühmann et al. (Foaming characteristics of oat protein and modification by partial hydrolysis, European Food Research and Technology, Vol. 244, n^{o}12, 28 Aug. 2018, pages 2095-2106) describes the production of an oat protein isolate using an oat protein concentrate obtained by supercritical CO₂ extraction according to EP2120604 as a starting material, using a step of alkaline extraction of this concentrate, a step of separation of the protein into the supernatant and a step of lyophilisation of this supernatant to produce the oat protein isolate powder.

The objective of the present patent application is to overcome these problems and thus to propose a new process that improves prior art existing techniques thereby delivering a unique oat protein powder.

### DESCRIPTION OF EMBODIMENTS

A first embodiment of the present invention is an oat protein composition characterized in that said composition does not contain traces of organic solvent does not contain traces of polysorbate, and preferably of any surfactant, has extractable lipid content below 10% by weight on dry matter based on the total dry weight of the oat protein composition and has a mean particle size greater than 10 microns.

A second embodiment is a process for producing an oat protein composition which has an extractable lipid content below 10% by weight on dry matter based on the total dry weight of the oat protein composition, which can be the oat protein composition of the present invention defined above, characterized in that the process comprises the following steps:
1) preparing a protein-rich suspension from oat starting material;
2) adding an amylase enzyme to the protein-rich suspension of step 1, thereby hydrolyzing the starch of the protein-rich suspension;
3) optionally, separating by centrifugation the protein-rich suspension of step 2 until obtaining a heavy layer comprising fibers and a light layer comprising proteins and collecting the light layer comprising proteins;
4) adjusting the pH of the protein-rich suspension of step 2 or the light layer comprising proteins of step 3 to a value comprised between 6 and 7, preferably around 6.5 and heating to 60° C., then allowing to cool to a temperature comprised between 20 to 30° C., preferably 25° C., for 60 min;
5) adjusting the pH to a value comprised between 4.5 and 5.5, preferably around 5;
6) separating by centrifugation into a heavy layer containing proteins and a light layer containing soluble compounds including lipids and collecting said heavy layer containing proteins as an oat protein composition;
7) optionally, subjecting said oat protein composition to ultra-high temperature treatment;
8) optionally, subjecting said oat protein composition to homogenization, and
9) optionally, drying said oat protein composition.

A third and last embodiment are industrial uses of oat protein compositions of the invention, preferably in food, feed, pharmaceutical and cosmetic fields.

The present invention will be better understood with the following detailed description.

### DESCRIPTION OF DETAILED EMBODIMENTS

A first embodiment of the present invention is an oat protein composition characterized in that said composition does not contain traces of organic solvent, does not contain traces of polysorbate, and preferably of any surfactant, has extractable lipid content below 10% by weight on dry matter based on the total dry weight of the oat protein composition and has a mean particle size greater than 10 microns.

By “oat protein composition”, it is meant a composition comprising oat protein as the only source of protein. In other terms, the oat protein composition does not comprise any protein that comes from another origin than oat.

By “a composition that does not contain traces of organic solvent” it is meant a composition that contains less than 100 ppm of solvent, preferably less than 10 ppm of organic solvent and more preferably a composition that does not contain organic solvent at all.

By “organic solvent”, it is meant solvent based on compounds that contain carbon. On the opposite, inorganic solvents which are allowed in this invention do not contain carbon. A typical inorganic solvent allowed in the present invention is water.

By “a composition that does not contain traces of polysorbate” it is meant a composition that contains less than 300 ppm of polysorbate, preferably less than 100 ppm of polysorbate and more preferably a composition that does not contain polysorbate at all.

By “a composition that does not contain traces of surfactant” it is meant a composition that contains less than 300 ppm of surfactant, preferably less than 100 ppm of surfactant and more preferably a composition that does not contain surfactant at all.

By “surfactant”, it is meant a compound that lowers the surface tension (or interfacial tension) between two liquids, between a gas and a liquid, or between a liquid and a solid. Surfactants may act as detergents, wetting agents, emulsifiers, foaming agents, or dispersants. A typical surfactant used in the field is polysorbate. Polysorbates are a class of emulsifiers used in cosmetic, pharmaceuticals and food preparations. Polysorbates are oily liquids derived from ethoxylated sorbitan (a derivative of sorbitol) esterified with fatty acids. Common brand names for polysorbates include Scattics, Alkest, Canarcel, and Tween. Common used polysorbate are Polysorbate 20 (polyoxyethylene (20) sorbitan monolaurate), Polysorbate 40 (polyoxyethylene (20) sorbitan monopalmitate), Polysorbate 60 (polyoxyethylene (20) sorbitan monostearate) and Polysorbate 80 (polyoxyethylene (20) sorbitan monooleate) (number following ‘polyoxyethylene’ refers to total number of oxyethylene —(CH2CH2O)— groups found in the molecule and number following ‘polysorbate’ is related to the type of fatty acid associated with the polyoxyethylene sorbitan part of the molecule). Preferably, the polysorbate is Polysorbate 80 (polyoxyethylene (20) sorbitan monooleate) also known as Tween 80.

“Oat” in the present application must be understood as a cereal plant belonging to the botanical genus Avena. This genus can be divided in wild and cultivated species which have been cultivated for thousands of years as a food source for humans and livestock. The cultivated species contain
Avena sativa—the most cultivated specie, commonly referred to as “oats”.
Avena abyssinica—the Ethiopian oat, native to Ethiopia, Eritrea, and Djibouti; naturalized in Yemen and in Saudi Arabia
Avena byzantina, a minor crop in Greece and Middle East; introduced in Spain, Algeria, India, New Zealand, South America, etc.
Avena nuda—the naked oat or hulless oat, which plays the same role in Europe as does A. abyssinicain Ethiopia. It is sometimes included in A. sativa and was widely grown in Europe before the latter replaced it. As its nutrient content is somewhat better than that of the common oat, A. nuda has increased in significance in recent years, especially in organic farming.
Avena strigosa—the lopsided oat, bristle oat, or black oat, grown for fodder in parts of Western Europe and Brazil.

In a preferred embodiment, oat protein composition is a protein concentrate, or a protein isolate. The oat protein composition can thus have around 50% by weight of protein or above, based on dry matter based on the total dry weight of the oat protein composition, for example from around 55 to 85%.

In the present application, “protein concentrate” must be understood as an oat protein composition which contains from 50% to 70%, by weight of protein on dry matter based on the total dry weight of the oat protein composition.

In the present application, “protein isolate” must be understood as an oat protein composition which contains more than 70%, generally more than 75%, preferably more than 80% by weight of protein on dry matter based on the total dry weight of the oat protein composition. The protein isolate can comprise less than 95%, generally less than 90% of protein on dry matter based on the total dry weight of the oat protein composition.

Various protocols can be used from prior art in order to quantify the protein content. In the present application, a preferred method to quantify the protein content consists of 1) determining the nitrogen content in the composition and 2) multiplying the nitrogen content by 6.25 factor (which represent the average quantity of nitrogen in protein). The nitrogen content can be determined by any suitable method in the art, such as the Kjeldhal method or by using a combustion analyzer. Preferably, the nitrogen content is determined by a combustion analyzer.

In the present application “protein” must be understood as molecules, consisting of one or more long chains of amino-acid residues. In the present application, proteins can be native proteins or modified proteins, including hydrolyzed proteins. These proteins can be present in different concentrations, including protein isolates or protein concentrates. Oats are the only cereal containing avenalin as globulin or legume-like protein, as the major storage protein (80% by weight). Globulins are characterized by their solubility in dilute saline as opposed to the more typical cereal proteins, such as gluten and zein which is a prolamine. The minor protein of oat is the prolamine which is called avenin.

In the present application “extractable lipid” must be understood as molecules that are soluble in nonpolar solvents for example petroleum ether, i.e. extractable lipids. Lipids include fatty acids, waxes, sterols, fat-soluble vitamins (such as vitamins A, D, E, and K), monoglycerides, diglycerides and triglycerides. Oats, after corn, have the highest lipid content of all the cereals, i.e. greater than 6%, sometimes greater than 10% by weight for some oats, in comparison to about 2-3% by weight for wheat and most other cereals.

One advantage of the present invention is even the lipids that are not soluble in non-polar solvents can also be eliminated, leading to an oat protein composition having a total lipid content which is also low.

The total lipid content used for the invention is acid hydrolysis using AOAC 996.06 method, while extractable lipid is measured by Soxhlet method using petroleum ether using AOAC 963.15 protocol.

The oat protein composition of the invention comprises an extractable lipid content below 10% by weight on dry matter based on the total dry weight of the oat protein composition, advantageously below 9%, more advantageously below 8%, even more advantageously below 7%, preferentially below 6%. The oat protein composition of the invention may comprises an extractable lipid content above 1% by weight on dry matter based on the total dry weight of the oat protein composition, for example more than 2%. The oat protein composition of the invention comprises an total lipid content below 10% by weight on dry matter based on the total dry weight of the oat protein composition, advantageously below 9%, more advantageously below 8%. The oat protein composition of the invention may comprises an total lipid content above 1% by weight on dry matter based on the total dry weight of the oat protein composition, for example more than 2%.

The oat protein composition can comprise from 0.1 to 10% by weight of starch on dry matter based on the total dry weight of the oat protein composition, preferably from 0.5 to 6%, more preferably from 1 to 4%. Starch content of the composition can be determined using AOAC Official Method 996.11, Starch (Total) in Cereal Products, and more particularly using the method of the booklet Megazyme, Total starch assay procedure (amyloglucosidase/α-amylase method) K-TSTA-50A/K-TSTA-50A 11/20, AOAC 996.11.

The oat protein composition can comprise a soluble fiber content going below 10% by weight on dry matter based on the total dry weight of the oat protein composition, preferably from 0.1 to 5%, more preferably from 0.1 to 3%. In the present application, soluble fiber content, insoluble fiber content, fiber content (which includes the total of soluble and insoluble fiber contents) can be determined using AOAC Official Method 2017.16, Total Dietary Fiber in Foods and Food Ingredients. By soluble fibers, it is meant to be fibers soluble in ethanol as described in this method. One of the dietary fibers generally present in the composition is beta-glucans.

The oat protein composition presents advantageously a mean particle size greater than 20 microns, preferably greater than 30 microns, more preferably greater than 40 microns. The oat protein composition presents advantageously a mean particle size lower than 300 microns, preferably lower than 200 microns, more preferably lower than 150 microns.

In the present application “particle size” must be understood as a notion introduced for comparing dimensions of solid, liquid or gaseous particles. The particle-size distribution (PSD) of a powder, or granular material, or particles dispersed in fluid, is a list of values or a mathematical function that defines the relative amount, typically by mass, of particles present according to size. Several methods can be used for measuring particle size and particle size distribution. Some of them are based on light, or on ultrasound, or electric field, or gravity, or centrifugation. The use of sieves is a common measurement technique. In the present application, the use of laser diffraction method is preferred. As for “mean particle size” (d 50) determined by laser diffraction, this mean particle size is a volume-weighted mean particle size. The man skilled in the art will be able to select a laser diffraction method allowing him to obtain an accurate mean particle size determination. An example of such method is indicated in the examples section.

In the present application, “dry matter” must be understood as the relative percentage by weight of solids based on total weight of the sample. Every well-known method can be used but desiccation method, which consists of estimating quantity of water by heating a known quantity of sample, is preferred.

The oat protein composition can comprise, based on the total weight of the proteins in the composition, less than 50% of proteins having a molecular weight of 10 kDa and less, advantageously less than 45%, less than 40%, less than 35% or less than 30%, preferably less than 10%.

In an embodiment, the oat protein composition comprises, based on the total weight of proteins in the composition:
from 0.5 to 30% of proteins having a molecular weight of 300 kDa and more, advantageously from 5 to 15%,
from 10 to 75% of proteins having a molecular weight of between 50 and 300 kDa, preferably from 30 to 75%, advantageously from 45 to 65%,
from 10 to 50% of proteins having a molecular weight of between 10 and 50 kDa, advantageously from 25 to 45%,
from 0.5 to 40% of proteins having a molecular weight of 10 kDa and less, preferably, from 0.5 to 20%, advantageously from 1 to 10%, the sum making 100%.

In an embodiment, the oat protein composition comprises, based on the total weight of proteins in the composition:
from 0.5 to 30% of proteins having a molecular weight of 300 kDa and more, advantageously from 5 to 15%,
from 30 to 75% of proteins having a molecular weight of between 50 and 300 kDa, advantageously from 45 to 65%,
from 10 to 50% of proteins having a molecular weight of between 10 and 50 kDa, advantageously from 25 to 45%,
from 0.5 to 20% of proteins having a molecular weight of 10 kDa and less, advantageously from 1 to 10%,
   the sum making 100%.

An advantage of this preferred embodiment of the invention is that the molecular weight of the oat protein composition is high, which can provide different protein functionalities compared to low molecular weight oat protein composition such as described e.g. in the document Brückner-Gühmann et al. These functionalities can depend on the process of manufacturing that is detailed hereafter.

The protein molecular weight (MW) distribution can be determined using Size Exclusion Chromatography.

To do so, it is possible to use the following method which is indicated as an example. Samples can be dissolved in 200 mM phosphate buffer, pH=7.6, vortexed for 1 minute initially and 10 minutes later and stored at 4 C over-night. The solutions are centrifuged at 7000 g for 10 minutes, the supernatant is measured for soluble protein content the next day, and the samples are diluted to 10 mg/mL with phosphate buffer. The samples are chromatographed using 2 SEC columns (400 and 300 Agilent Advanced Bio SEC Column, 5000-1,250,000 MW Range) in sequence using phosphate buffer, pH=7.6 as the mobile phase at 0.5 mL/minute. The detection is a UV=280 nm. Several protein molecular weight standards going from 14300 to 669000 Da (Lysozyme, Carbonic Anhydrase, BSA, HSA, B-Amylase, Apoferritin, Thyroglobulin) are analyzed to identify the retention time and calibrate the chromatography apparatus. For sample analysis, chromatograms peak or peak apex (group) is determined along with the range of the peak (start and end) and the molecular weight is determined for the range and peak apex. The percent of molecular weight can be determined, for example, for: >300 kDa, 300 kDa to 50 kDa, 50 KDa to 10 KDa and <10 kDa.

In one embodiment, the oat protein composition can be hydrolysed. Hydrolysis can conducted by any means known, for example by using a protease or a peptidase enzyme.

A second embodiment of the present invention is a process for producing an oat protein composition which has a extractable lipid content below 10% by weight on dry matter based on the total dry weight of the oat protein composition, which can be the oat protein composition as defined above, characterized in that the process comprises the following steps:
1) preparing a protein-rich suspension from oat starting material;
2) adding an amylase enzyme to the protein-rich suspension of step 1, thereby hydrolyzing the starch of the protein-rich suspension;
3) optionally, separating by centrifugation the protein-rich suspension of step 2 until obtaining a heavy layer comprising fibers and a light layer comprising proteins and collecting the light layer comprising proteins;
4) adjusting the pH of the protein-rich suspension of step 2 or the light layer comprising proteins of step 3 to a value comprised between 6 and 7, preferably around 6.5 and heating to 60° C., then allowing to cool to a temperature comprised between 20° C. and 30° C., preferably 25° C., for 60 min;
5) forming a protein precipitate;
6) separating by centrifugation into a heavy layer containing proteins and a light layer containing soluble compounds including lipids and collecting said heavy layer containing proteins as an oat protein composition;
7) optionally, subjecting said oat protein composition to ultra-high temperature treatment;
8) optionally, subjecting said oat protein composition to homogenization, and
9) optionally, drying said oat protein composition.

Thus, the process of the present invention does not use organic solvents and allows obtaining a composition which does not contain traces of organic solvent.

Advantageously, it also does not use polysorbate or any other surfactant and allows obtaining a composition which does not contain traces of polysorbate and preferably of any surfactant.

The first step consists in preparing a protein-rich suspension from oat starting material. The oat starting material may comprise oat flour, low-fiber oat flour, oat bran or oat pulp fraction from oat milk production or oat syrup production. The oat starting material typically comprises protein, lipid, fiber and starch. The oat starting material does typically not comprise organic solvents because it is not preliminary treated with these.

Typically, the oat starting material comprises between 5 and 45% protein, between 5 and 80% starch, between 5 and 50% fiber and 3 to 15% extractable lipids, the amounts being expressed as weight % of each component based on the total dry solids of each material, the total amounting to 100%.

Typically, whole oat flour comprises 8-30% protein, 40-80% starch and 5-15% fiber and 3 to 15% extractable lipids.

Typically, low-fiber oat flour (or de-hulled oat flour) comprises 10-30% protein, 45-80% starch, 0 to 4% of fiber and 3-15% extractable lipids.

Typically, oat bran comprises 10-30% protein, 30-70% starch and 10-20% fiber and 4 to 15% extractable lipids.

Typically oat processed material (the oat pulp fraction which is a by-product from oat milk or oat syrup production) comprises 10-45% protein, 5-30% starch and starch hydrolysate and 10-50% fiber and 3 to 15% extractable lipids.

The protein-rich suspension is a suspension of oat flour in water. As water, any food compatible water can be used, but tap water, reverse osmosis water and deionized water are preferred. The aim of step 1 is to reach a dry matter comprised between 5% and 20%, preferably between 10% and 15%, most preferably between 10% and 13% by weight with respect to the total weight of the suspension.

Depending on the oat starting material, this protein-rich suspension can be obtained in different ways.
a) When oat seeds are used, said oat seeds may be cultivated and/or commercially available. Oat seeds may then prepared including possible steps of sieving or dehulling.

Oats seeds may be dry- or wet-heated prior to use. The purpose of dry- or wet-heat is to destroy enzymes including beta-glucanase, lipase and lipoxygenase. Indeed, inactivation of lipase and lipoxygenase is indicated to prevent the product from turning rancid. In the process of the present invention, heat treatment, in particular steaming, should be avoided or at least be kept as short as possible and/or carried out at a temperature as low as possible to keep oat protein denaturation low.

The oat seeds are then ground in order to obtain protein rich flour. To grind oat seeds, all well-known common technologies can be used including stone-mill, roller mill or knife-mill. In this step, preferred particle size distribution of the resulting protein rich flour may be a d50 (50th percentile) above 30 microns, preferably above 40 microns, even more preferably above 50 microns. In the present invention, d50 is measured with help of any known by man skilled in the art technology. In a preferred way, laser granulometry is preferred.

The protein rich flour can comprise a protein content above 14%, e.g. above 16%, based on the dry solids content of the protein flour.

Preferably, the content of insoluble fiber in the protein rich flour is less than 4%, preferably less than 2%, based on the dry solids content of the protein flour. In these preferred ranges, the viscosity is lower during the process, which makes easier the conduction of the process.
b) It is possible to use directly commercial oat flour. When oat flour is used, typically, the flour may be weighed and introduced in a tank containing water and equipped with agitation, pH and heating apparatus at the desired dry matter content.
c) It is also possible to prepare the suspension from oat bran. Oat bran is made up of only the outer shells of the seed. When oat seed is processed to remove the inedible exterior body of the seed, this leaves behind the oat groat, and oat bran is the outer layer of this oat groat kernel, which is right underneath the inedible grain portion.
d) it is also possible to prepare the suspension of step 1 from oat pulp. Oat pulp, also known as oat okara, is the pulp that is generally discarded during the preparation of oat milk or oat syrup. Briefly, the procedure starts by measuring and milling the oat grains to break apart their outer hull. Then the grains are stirred in warm water and ground into a slurry. The slurry is treated with enzymes and heat to create a thick liquid oat base, which is then separated by decanting, filtering, or centrifugation. The supernatant represents the oat milk, whereas the pellet is the oat pulp or okara.

Preferably, during step 1, the temperature is regulated between 60° C. to 80° C., preferably between 65° C. and 75° C. Preferably, during step 1 pH is adjusted between 5 and 6, preferably 5.5. In the present application, pH can be adjusted by adding well-known acid or basic compounds such as hydrochloric acid, sodium hydroxide, citric acid, calcium hydroxide and potassium hydroxide. Agitation may be set-up in order to obtain a homogeneous suspension, without foaming.

The second step aims to hydrolyze the starch contained in the protein-rich suspension with the help of amylases. Amylases are type of enzymes that catalyzes hydrolysis of starch molecules in smaller sugar molecules. Any type of amylase can be used like beta-amylases or amyloglucosidase, but alpha-amylases are preferred. In a preferred embodiment, thermoresistant alpha-amylases are preferred.

The aim of step 2 is to efficiently reduce the size of starch contained in the protein-rich suspension by hydrolysis, thereby obtaining a soluble dextrin or glucose syrup instead of starch. This soluble transformation of starch will allow a more simple separation with insoluble compounds in the coming steps.

In a preferred embodiment, alpha-amylase enzyme is preferred. Activity of alpha-amylase is expressed as KNU units. In practice, the α-amylase activity is measured using ethylidene-G7-PNP (4,6-ethylidene(G7)-p-nitrophenyl(G1)-α,D-maltoheptaoside) as a substrate. The compound is hydrolyzed by the LE399 alpha-amylase to G2-PNP and G3-PNP where G means glucose and PNP means p-nitrophenol. G2-PNP and G3-PNP are subsequently hydrolyzed by α-glucosidase, which is added to the reaction mixture, to glucose and p-phenol. Absorbance is measured spectrophotometrically at 409 nm under standard reaction conditions. One KNU(T) corresponds to the amount of αalpha-amylase that hydrolyzes 672 micromoles of ethylidene-G7PNP per minute under standard conditions (pH 7.1; 37° C. The quantification limit of the method is approximately 0.3 KNU(T)/g. In step 2, the amylase may be added in an amount having an activity level comprised between 10 and 170 KNU/100 g of flour, preferably between 50 and 160 KNU/100 g of flour, even more preferably between 100 and 150 KNU/100 g of flour. One Kilo Novo alpha-amylase Unit (KNU) is a value known by the man skilled in the art and is the amount of enzyme which breaks down a determined quantity of starch per hour at Novozymes' standard method. This test consists in determining alpha-amylase activity relative to an alpha-amylase standard with known activity (Termamyl) and is expressed in Kilo Novo alpha-amylase Units (KNU). One KNU is the amount of alpha-amylase which, under standard conditions (pH 7.1; 37° C.), dextrinizes 5.26 g starch dry substance per hour.

The suspension has, at least during a part of the step 2), a pH going from 1.5 to 3.0 or from the range 7.0 to 11.0, preferably 2.0-2.5 or 8.5-10.5, even more preferable 8.5-10.5. At alkaline pH, the protein recovery is even higher than in the case of acid extraction. The solvent is preferably water. However, it can be added compounds to make higher the solubility of the protein fraction in the suspension from oat-processed material. Any inorganic or organic acid and base reactant can be used. They may be chosen from caustic soda, potash, lime, citric acid, ascorbic acid, nitric acid, sulfuric acid and hydrochloric acid. The pH of the protein-rich suspension can be set with these acid and base components to a pH going from 1.5 to 3.0 or from the range 7.0 to 11.0, preferably 2.0-2.5 or 8.5-10.5. The temperature during that steps a and b can be adjusted between 2 and 80° C., for example between 10° C. and 30° C.

The temperature during the step 2) can also be adjusted between 10 and 180° C. In an embodiment, the temperature during step b is adjusted so that the suspension has a temperature comprised between 10° C. and 80°, preferably between 30° and 70° C., for at least part of step b. In another embodiment, the temperature can be brought to a temperature from 80 to 180° C., for example from 100 to 155° C. for at least part of step b. The step b) can have a duration, depending on the temperature, from 1 s to 90 minutes. For example, when the temperature is from 100 to 155° C., the duration may be from 5 to 90 s. For example, when the temperature is between 80 and 100° C., the duration may be from 30 seconds to 20 minutes. For example, when the temperature is between 30 and 80° C., the duration may be from 5 minutes to 90 minutes.

The third step, which is optional, consists in a centrifugation in order to separate a heavy layer comprising fibers and a light layer comprising proteins. Indeed, as fibers and residual starch are insoluble and heavier than proteins, sugar and salts, they will be separated with help of a centrifuge. This step is not required when the amount of dietary fiber present in the oat starting material is relatively low.

More than 70% by weight of the dry matter, preferably more than 80% by weight of the dry matter obtained after optional step 3 is constituted of proteins.

The fourth step of the method according to the invention consists in adjusting the pH to a value comprised between 6 and 7, preferably around 6.5, and heating to a temperature from 50 to 80° C., preferably from 50 to 70° C., from 55 to 65° C., even more preferably around 60° C. The mixture is then allowed to cool to a temperature comprised between 20° C. and 30° C., preferably around 25° C. This step typically lasts around 15 to 240 minutes, preferably between 30 minutes to 90 minutes, even more preferably around 60 minutes.

In a fifth step, a proteic precipate is formed. This step may be done by adjusting the pH of the heated additive-containing soluble fraction close to isoelectric point, for example in the range going from 4.5 to 5.8 to form a proteic precipitate, preferably from 5.0 to 5.5. This step can be done at a temperature going from 20 to 80° C., preferably at a temperature going from 50 to 60° C.

The sixth step consists in a centrifugation allowing separation into a heavy layer (which contains mainly proteic precipitate) and a light layer containing others compounds including proteic precipitate. The pellet, lower part, underflow or heavy layer, which contains proteins, is collected. The supernatant, higher layer overflow or light layer, which contains hydrolyzed starch and lipids, is discarded.

In a preferred embodiment, the pellet, lower part or heavy layer is mixed with water, agitated and then fed in a second centrifuge. Once again, the pellet, lower part or heavy layer, which contains proteins is collected. The supernatant, higher layer or light layer, which contains hydrolyzed starch and lipids is discarded.

The resulting product is an oat protein composition according to the present invention.

In a seventh, optional step, the oat protein is subjected to sterilization by ultra-high temperature treatment.

In an eighth, optional step, the oat protein composition is subjected to homogenization.

In a ninth, optional step, the oat protein composition can be dried. In order to do so, man skilled in the art may preferably use a spray-drier, preferably a multistage spray-drier.

This will allow obtaining an oat protein composition having the mean particle size defined above.

Typical spray drying parameters range are 180-220° C. inlet air temperature; and 80-110° C. outlet temperature, in order to produce oat protein compositions in the form of a powder having less than 5% moisture.

The process according to the invention may also comprise a step of modifying the proteins in the oat protein composition by subjecting the composition to enzymatic treatment with a protease or a glutaminase. This step may be carried out preferably at any stage after step 6) (which allows the collecting of the proteins).

A third and last embodiment of the present invention is the use of the oat protein composition of the present invention or obtained by the process of the present invention, preferably in food, feed, pharmaceutical and cosmetic fields.

Such oat protein composition is particularly suitable for ready to drink beverages, or baking or any other food application such as protein bars, non-dairy beverages, powder mixes, yogurts, cheeses, or meat-like products. Its low lipid content allows an improved organoleptic experience when formulated, Indeed, when a product comprises high amounts of lipids, these undesirable lipids can get oxidized and develop a rancid taste, which negatively affects the organoleptic quality of the product.

Such oat protein composition is particularly suitable for ready to drink beverages or baking or any other food application such as protein bars, non-dairy beverages, powder mixes, yogurts, cheeses, or meat-like products. Its low lipid content allows an improved organoleptic experience when formulated, Indeed, when a product comprises high amounts of lipids, these undesirable lipids can get oxidized and develop a rancid taste, which negatively affects the organoleptic quality of the product.

In general terms, the oat protein composition of the invention can be used in food and beverage products that may include the oat protein composition in an amount of up to 100% by weight relative to the total dry weight of the food or beverage product, for example in an amount of from around 1% by weight to around 80% by weight relative to the total dry weight of the food or beverage product. All intermediate amounts (i.e. 2%, 3%, 4% . . . 77%, 78%, 79% by weight relative to the total weight of the food or beverage product) are contemplated, as are all intermediate ranges based on these amounts.

Beverages include acid beverages, carbonated beverages (including, but not limited to, soft carbonated beverages); non-carbonated beverages (including, but not limited to, soft non-carbonated beverages such as flavored waters, fruit juice and sweet tea or coffee based beverages); beverage concentrates (including, but not limited to, liquid concentrates and syrups as well as non-liquid ‘concentrates’, such as freeze-dried and/or powder preparations). The protein content in the beverage can be very different and the beverage can be a high protein drink. The content is for example between 1 and 12% of the total mass of the beverage, for example between 2 and 10%. Beverages also include milk-like beverages, that can be «barista» type or «coffee creamer» type.

Food products which may be contemplated in the context of the present invention include baked goods; sweet bakery products (including, but not limited to, rolls, cakes, pies, pastries, and cookies); pre-made sweet bakery mixes for preparing sweet bakery products; pie fillings and other sweet fillings (including, but not limited to, fruit pie fillings and nut pie fillings such as pecan pie filling, as well as fillings for cookies, cakes, pastries, waffles, pancakes, muffins and biscuits, confectionary products and the like, such as fat-based cream fillings); desserts such as flan, custard, gelatins and puddings; frozen desserts (including, but not limited to, frozen dairy desserts such as ice cream—including regular ice cream, soft serve ice cream and all other types of ice cream—and frozen non-dairy desserts such as non-dairy ice cream, sorbet and the like); snack bars (including, but not limited to, cereal, nut, seed and/or fruit bars); bread products (including, but not limited to, leavened and unleavened breads, yeasted and unyeasted breads such as soda breads, breads comprising any type of wheat flour, breads comprising any type of non-wheat flour (such as oat, potato, rice and rye flours), gluten-free breads); pre-made bread mixes for preparing bread products; sauces, syrups and dressings; sweet spreads (including, but not limited to, jellies, jams, butters, nut spreads, dulce de leche and other spreadable preserves, conserves and the like); confectionary products (including, but not limited to, jelly candies, soft candies, hard candies, chocolates, caramels and gums); sweetened and un sweetened breakfast cereals (including, but not limited to extruded breakfast cereals, flaked breakfast cereals and puffed breakfast cereals); and cereal coating compositions for use in preparing sweetened breakfast cereals. Other types of food and beverage product may also be contemplated in the context of the present invention. In particular, animal foods (such as pet foods) are explicitly contemplated.

Oat protein can be used in combination with flavours or masking agents.

Oat protein can also be used, eventually after texturization, in meat-like products such as emulsified sausages or plant-based burgers, fish-like products or seafood-like products. It can also be used for making egg substitutes or for the manufacturing of protein containing products such as tofu or tempeh. «Texturized proteins» generally means proteins texturized by extrusion, i.e. especially by dry extrusion to make Textured Vegetable Protein, wet extrusion or high moisture extrusion. Extruders can be single screw extruders, twin screw extruders, multiple screw extruders. Example of multiple screw extruders are planetary extruder or ring-extruder. Other technologies such as shear cell technology, microextrusion or 3D printing can also be used.

The food or beverage product can be used in specialized nutrition, for specific populations, for example for baby or infants, teenagers, adults, elderly people, athletes, people suffering from a disease. It can be meal substitutes formulations, complete nutrition beverages, for example for weight management or in clinical nutrition (for example tube feeding or enteral nutrition).

The oat protein composition can be used as the sole source of protein but also can be used in combination with other plant or animal proteins. These other proteins can be hydrolyzed or not. Generally, these are in the form of isolates or concentrates. The term “plant protein” denotes all the proteins derived from cereals, oleaginous plants, leguminous plants and tuberous plants, and also all the proteins derived from algae and microalgae or fungi, used alone or as a mixture, chosen from the same family or from different families. In the present application, the term “cereals” is intended to mean cultivated plants of the grass family producing edible grains, for instance wheat, rye, barley, maize, sorghum or rice. The cereals are often milled in the form of flour, but are also provided in the form of grains and sometimes in whole-plant form (fodders). In the present application, the term “tubers” is intended to mean all the storage organs, which are generally underground, which ensure the survival of the plants during the winter season and often their multiplication via the vegetative process. These organs are bulbous owing to the accumulation of storage substances. The organs transformed into tubers can be the root e.g. carrot, parsnip, cassava, konjac), the rhizome (e.g. potato, Jerusalem artichoke, Japanese artichoke, sweet potato), the base of the stalk (more specifically the hypocotyl, e.g. kohlrabi, celeriac), the root and hypocotyl combination (e.g. beetroot, radish). For the purposes of the present invention, the term “leguminous plants” is intended to mean any plants belonging to the family Cesalpiniaceae, the family Mimosaceae or the family Papilionaceae, and in particular any plants belonging to the family Papilionaceae, for instance pea, bean, soy, broad bean, horse bean, lentil, alfalfa, clover or lupin. This definition includes in particular all the plants described in any of the tables contained in the article by R. Hoover et al., 1991 (Hoover R. (1991) “Composition, structure, functionality and chemical modification of legume starches: a review” Can. J. Physiol. Pharmacol., 69, pp. 79-92). Oleaginous plants are generally seed-producing plants from which oil is extracted. Oilseed plants can be selected from sunflower, rapeseed, peanut, sesame, pumpkin or flax. The animal protein can be for example egg or milk proteins, such as whey proteins, casein proteins or caseinate. The oat protein composition can thus be used in combination with one or more of these proteins or amino acids in order to improve the nutritional properties of the final product, for example to improve the PDCAAS of the protein or to bring other or modify functionalities.

The oat protein can also be used for the manufacturing of pharmaceutical products or in fermentation, for example for the production of fungi metabolites or cell culture metabolites.

The oat protein composition of the invention can also be used for acidic food products such as yogurts (including, but not limited to, full fat, reduced fat and fat-free dairy yogurts, as well non-dairy and lactose-free yogurts and frozen equivalents of all of these), cheeses or acidic sauces. Acidic food products can have a pH of 3 to 6 when diluted at a dry matter of 10%. The oat protein composition can be used to form of a milk and fermented and/or acidified to provide yogurts and cheeses. These milks can present a dry matter going from 5 to 30%. These milks can comprise other components such as sugars and fats and optional, Yogurts can include stirred yogurts, set yogurts or yogurts to drink. These can be flavoured or not and can include other components such as fruit preparations and/or sweeteners. Cheeses can be process cheese, swiss cheese, string cheese, ricotta, provolone, parmesan, muenster, mozzarella, jack, manchego, blue, fontina, feta, edam, double Gloucester, cheddar, asiago and Havarti. Acidic sauces are for example mayonnaise or ketchup.

The invention will be better understood with the following non-exhaustive examples and figures.

### EXAMPLES

### Example 1: Analytical Methods

Method for Determining Protein Content:
% protein: Protein content is % N6.25 and nitrogen content is determined using combustion analyzer-Elementer, with AOAC 997.09 method.
% lipids: extractable lipid content is determined using Soxhlet extraction in petroleum ether using AOAC 963.15 method and total lipid content is determined using AOAC 996.06 method.
% starch: AOAC Official Method 996.11.
% soluble fiber: AOAC official method 2017.16.
% insoluble fiber: AOAC official method 2017.16.
Color L*a*b*: Determined using a device CR-5 from Konica Minolta following the instructions manual.
% Moisture: Weigh 2-3 grams of sample into pre-weighed aluminum drying pan. Place in 130° C. oven for 2 hours, cooled to room temperature, weigh samples again. Moisture determined based on mass loss.
Protein recovery: mass of protein in the isolate obtained before spray drying/mass of protein in the flour×100
d 50 (μm): d 50 is measured by a laser granulometry apparatus (Mastersizer 3000, from Malvern), which measures intensity of scattered light across a range of scattering angles using forward scattering measurement, on a dry powder without dispersion buffer, and using the software of the apparatus with the Mie scattering model to fit the distribution to the measured scattering pattern.
Spray drying yield: mass of dry powder obtained/mass of dry composition injected×100

### Example 2: Production of Oat Protein Composition at Pilot Scale

The protein composition was produced using the following protocol:

A 50 gall (189 L) jacketed tank was filled with approx. 160 L of 50° C. water. One bag of 50 lb (22.68 kg) of low-fiber oat flour N^{o}70 from Grain Millers was mixed into water and adjusted to achieve 10.5% (+/−0.5%) solids. The pH was adjusted to 5.4 to 5.5 with HCl while agitating for 10 min. 230 g of Liquozyme supra (from Novozyme) was added and the mixture was heated to 70° C., recirculated with a lobe pump for 2 hours.

Following this enzymatic treatment, the pH was adjusted to 7.0 with NaOH and the mixture was centrifuged at 1500 g with a Lemitec decanter centrifuge, (5000 rpm, 10 rpm diff, 2000 ml/min feed, with 60/10 weir). The overflow (OF) was collected in 100 gal (278.5 L) jacketed tank, and held at 50° C. with hot water on tank jacket.

The mixture was heated to 65° C., the pH adjusted to 6.5, recirculating with centrifugal pump at 30 Hz. The mixture was then cooled to 25° C. using cool water on jacket while agitating over 20-30 minutes (example according to the invention). The mixture was recirculated with centrifugal pump for a total of 60 minutes.

Following this step, the separation step was carried out by filling the tank to capacity with ambient temperature water, reducing the pH to 5.0 with HCl and centrifuging on a stacked disc centrifuge (Clara 20, 0.45 m\hat{ }3/h, 9,000 rpm).

The underflow fraction (UF) was collected and resuspended in a jacketed 100 gal (278.5 L) tank, filled to capacity with ambient temperature water. The pH was adjusted to pH to 5.0 with HCl and the % volume solids measured.

The composition was subjected to centrifugation on a stacked disc (Clara 20, 0.45 m\hat{ }3/h, 9,000 rpm). This UF fraction was stored overnight in a 4-8° C. fridge.

The curd was warmed to 25° C., the pH adjusted to pH 9.3 and held for 30 min. It was then passed through Ultra High Temperature sterilization (UHT) at 300-310° F. (149 to 154° C.) hold temp, 160° F. (71° C.) flash temp, 30 s hold time.

After adjusting the pH to 6.8 at 60-65° C., the composition was homogenized at 400 bar 1^{st} stage pressure, 40 bar 2^{nd} stage, and spray-dried at 220° C. inlet, 90° C. outlet.

The resulting oat protein composition according to the invention comprised 78.8% protein, 3.6% lipids, 5.2% of insoluble fiber, 5.9% of soluble fiber, 5.0% of beta-glucans and 3.5% of moisture. The starch content is determined to be around 1.3%.

The same experiment was carried out with the following variations concerning the holding step at pH 6.5 prior to separation of the protein-rich fraction and lipid-rich fraction:
addition of Tween 80 and holding at 65° for 60 min (comparative example 1);
holding at 65° for 60 min (comparative example 2);
no hold (comparative example 3)

The amounts of protein and lipid present in the oat protein composition obtained under each condition are summarized in Table 1 below:

| | “pH 6.5 hold” | | % extractable |
|---|---|---|---|
| | condition | % protein | lipid |
| Comparative | Tween 80 + hold | 80% | 3-5% |
| Example 1 | at 65° for 60 min | | |
| Comparative | Hold at 65° | 73.3% | 13.3% |
| Example 2 | for 60 min | | |
| Comparative | No hold | 78% | 13% |
| Example 3 | | | |
| Example according | Hold at 65-25° | 79% | 4% |
| to the invention | C. for 60 min | | |

As can be seen in Table 1, comparative example 1 gave satisfactory results in terms of protein and lipid percentages.

When the same experiment was carried out without Tween 80, with a separation step was carried at 60°, with no prior cooling to 25° C. (comparative example 2), the resulting oat protein composition comprised 13.3% lipids by weight. This amount of lipids is not satisfactory.

When the hold step at pH 6.5 was omitted entirely, the resulting product also contained 13% of lipids, which is not satisfactory.

In conclusion, the inventors have demonstrated that, unexpectedly, including a step wherein the material is held at a pH around 6.5, heated to 65° and then cooled to 25° C., prior to the separation step by centrifugation, allows an efficient separation between the lipids and the proteins.

The average molecular weight of the proteins obtained according to the invention in Example 2 was determined to be 35602 g/mol.

The following distribution was determined by size exclusion chromatography:
10.7% of proteins having a molecular weight of 300 kDa and more,
11.84% of proteins having a molecular weight of between 50 and 300 kDa,
41.05% of proteins having a molecular weight of between 10 and 50 kDa,
36.04% of proteins having a molecular weight of 10 kDa and less.

1. Oat protein composition characterized in that said composition does not contain traces of organic solvent and does not contain traces of polysorbate, and preferably of any surfactant, has extractable lipid content below 10% by weight on dry matter based on the total dry weight of the oat protein composition and has a mean particle size (d 50), determined by laser diffraction, greater than 10 microns.

2. The oat protein composition according to claim 1 wherein said composition contains from 40% to 70%, preferably from 50% to 60% by weight of protein on dry matter based on the total dry weight of the oat protein composition.

3. The oat protein composition according to claim 1 wherein said composition contains more than 70%, preferably more than 80% by weight of protein on dry matter based on the total dry weight of the oat protein composition.

4. The oat protein composition as defined in claim 1 wherein said composition comprises, based on the total weight of proteins in the composition:
from 0.5 to 30% of proteins having a molecular weight of 300 kDa and more, advantageously from 5 to 15%,
from 30 to 75% of proteins having a molecular weight of between 50 and 300 kDa, advantageously from 45 to 65%,
from 10 to 50% of proteins having a molecular weight of between 10 and 50 kDa, advantageously from 25 to 45%,
from 0.5 to 20% of proteins having a molecular weight of 10 kDa and less, advantageously from 1 to 10%,
the sum making 100%.

5. The oat protein composition according to claim 1, wherein the composition comprises from 0.1 to 10% by weight of starch on dry matter based on the total dry weight of the oat protein composition, preferably from 0.5 to 6%, more preferably from 1 to 4%.

6. The oat protein composition according to claim 1, wherein the composition comprises a total dietary fiber going from 0.1 to 10% by weight of fiber on dry matter based on the total dry weight of the oat protein composition, preferably from 0.5 to 6%, more preferably from 1 to 4%.

7. The oat protein composition according to claim 1, wherein the composition presents:
a mean particle size greater than 20 microns, preferably greater than 30 microns, more preferably greater than 40 microns, and
a mean particle size lower than 300 microns, preferably lower than 200 microns, more preferably lower than 150 microns.

8. Process for producing an oat protein composition which has an extractable lipid content below 10% by weight on dry matter based on the total dry weight of the oat protein composition characterized in that the process comprises the following steps:
1) preparing a protein-rich suspension from oat starting material;
2) adding an amylase enzyme to the protein-rich suspension of step 1, thereby hydrolyzing the starch of the protein-rich suspension;
3) optionally, separating by centrifugation the protein-rich suspension of step 2 until obtaining a heavy layer comprising fibers and a light layer comprising proteins and collecting the light layer comprising proteins;
4) adjusting the pH of the protein-rich suspension of step 2 or the light layer comprising proteins of step 3 to a value comprised between 6 and 7, preferably around 6.5 and heating to 50 to 80° C., preferably 50 to 70° C., 55 to 65° C., even more preferably around 60° C., then allowing to cool to a temperature comprised between 20 to 30° C., preferably 25° C., over a period of time between 15 and 240 minutes, preferably between 30 minutes and 90 minutes, even more preferably around 60 minutes;
5) forming a protein precipitate;
6) separating by centrifugation into a heavy layer containing protein precipitate and a light layer containing soluble compounds including lipids and collecting said heavy layer containing proteins as an oat protein composition;
7) optionally, subjecting said oat protein composition to ultra-high temperature treatment;
8) optionally, subjecting said oat protein composition to homogenization, and
9) optionally, drying said oat protein composition.

9. Process according to claim 8 wherein the amylase enzyme of step 2 is a thermo-resistant amylase.

10. Process according to claim 8 wherein the amylase added in step 2 has an activity level comprised between 10 and 170 KNU/100 g of flour, preferably between 50 and 160 KNU/100 g of flour, even more preferably between 100 and 150 KNU/100 g of flour.

11. Process according to claim 8 wherein the oat protein composition is as defined.

12. Use of the oat protein composition as defined in claim 1, in food, feed, pharmaceutical and cosmetic fields.

13. Use of the oat protein composition obtained according to the process defined in claim 8, in food, feed, pharmaceutical and cosmetic fields.
